(19) United States
(12) Patent Application Publication
(10) Pub. No.: US 2008/0200513 A1
(43) Pub. Date: Aug. 21, 2008

(54) Pyridine N-Oxides As Antiviral Agents

(75) Inventors: Stafania Colarusso, Rome (IT); Frank Narjes, Rome (IT)

Correspondence Address: MERCK AND CO., INC, P O BOX 2000, RAHWAY, NJ, 07065-0907, US

(73) Assignee: ISTITUTO DI RICERCHE DI BIOLOGIA MOLECOLARE P ANGELETTI SPA, Pomezia (Rome) (IT)

(21) Appl. No.: 10/556,965

(22) PCT Filed: Jun. 1, 2004
(86) PCT No.: PCT/EP2004/005971

§ 371 Date: Apr. 4, 2008

(30) Foreign Application Priority Data
Jun. 9, 2003 (GB) 0313250.3

(51) Int. Cl. A61K 31/4436; A61K 31/4412; A61P 31/14; C07D 213/78; C07D 409/04

(52) U.S. Cl. 514/336; 546/298; 546/280.4; 514/345

(57) ABSTRACT

The present invention relates to pyridinone derivatives of formula (I):

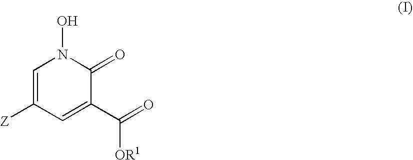

wherein Z represents C₂₋₆ alkynyl, aryl or heteroaryl, any of which groups may be optionally substituted, and R¹ represents hydrogen, C₁₋₆ alkyl, C₃₋₇ heterocycloalkyl(C₁₋₆)alkyl, di(C₁₋₆)alkylamino(C₁₋₆)alkyl, C₂₋₆ alkylcarbonyloxy(C₁₋₆)alkyl or C₃₋₇ cycloalkoxycarbonyloxy(C₁₋₆)alkyl, and pharmaceutically acceptable salts thereof, useful in the prevention and treatment of hepatitis C virus infections.

This invention relates to compounds which can act as inhibitors of viral polymerases, especially the hepatitis C virus (HCV) polymerase, to uses of such compounds and to their preparation.

The hepatitis C virus (HCV) is the major causative agent of parenterally-transmitted and sporadic non-A, non-B hepatitis (NANB-H). Some 1% of the human population of the planet is believed to be affected. Infection by the virus can result in chronic hepatitis and cirrhosis of the liver, and may lead to hepatocellular carcinoma. Currently no vaccine nor established therapy exists, although partial success has been achieved in a minority of cases by treatment with recombinant interferon-α, either alone or in combination with ribavirin. There is therefore a pressing need for new and broadly-effective therapeutics.

Several virally-encoded enzymes are putative targets for therapeutic intervention, including a metalloprotease (NS2-3), a serine protease (NS3), a helicase (NS3), and an RNA-dependent RNA polymerase (NS5B). Of these, the polymerase plays an essential role in replication of the virus and is therefore an important target in the fight against hepatitis C.

It has now been found that certain pyridinone derivatives act as inhibitors of hepatitis C virus (HCV) polymerase enzyme.

The present invention provides a compound of formula (I) below, or a pharmaceutically acceptable salt thereof:

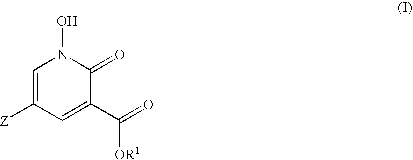

wherein

Z represents C₂₋₆ alkynyl, aryl or heteroaryl, any of which groups may be optionally substituted; and

R¹ represents hydrogen, C₁₋₆ alkyl, C₃₋₇ heterocycloalkyl(C₁₋₄)alkyl, di(C₁₋₆)alkylamino(C₁₋₆)alkyl, C₂₋₆ alkylcarbonyloxy(C₁₋₆)alkyl or C₃₋₇ cycloalkoxycarbonyloxy(C₁₋₆)alkyl.

It will be appreciated that the compound of formula (I) as depicted above may exist in equilibrium with its other tautomeric forms, including in particular the structure of formula (IA):

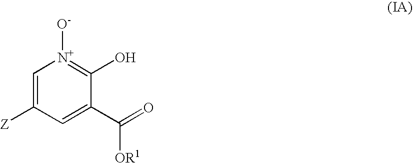

wherein Z and R¹ are as defined above. It is to be understood that all tautomeric forms of the compounds of formula (I), as well as all possible mixtures thereof in any proportion, are included within the scope of the present invention.

The present invention also provides a compound of formula (I) as defined above, or a tautomer thereof, or a pharmaceutically acceptable salt thereof, for use in therapy, especially for pharmaceutical use in humans.

Typical examples of C₁₋₆ alkyl groups include methyl and ethyl groups, and straight-chained or branched propyl, butyl, pentyl and hexyl groups. Particular alkyl groups are methyl, ethyl, n-propyl, isopropyl, tert-butyl and 1,1-dimethylpropyl. Derived expressions such as “C₁₋₆ alkoxy” are to be construed accordingly.

Typical examples of C₂₋₆ alkenyl groups include vinyl, allyl and dimethylallyl groups.

Typical examples of C₂₋₆ alkynyl groups include ethynyl and propargyl groups.

Typical C₃₋₇ cycloalkyl groups include cyclopropyl, cyclobutyl, cyclopentyl and cyclohexyl.

Suitable C₃₋₇ heterocycloalkyl groups include azetidinyl, pyrrolidinyl, piperidinyl, piperazinyl, morpholinyl and thiomorpholinyl groups.

Suitable aryl groups include phenyl and naphthyl, especially phenyl.

Suitable heteroaryl groups include pyridinyl, quinolinyl, isoquinolinyl, pyridazinyl, pyrimidinyl, pyrazinyl, furyl, benzofuryl, dibenzofuryl, thienyl, benzthienyl, pyrrolyl, indolyl, pyrazolyl, indazolyl, oxazolyl, isoxazolyl, thiazolyl, isothiazolyl, imidazolyl, benzimidazolyl, oxadiazolyl, thiadiazolyl, triazolyl and tetrazolyl groups.

Typical aryl(C₁₋₆)alkyl groups include benzyl, phenylethyl, phenylpropyl, phenylbutyl and naphthylmethyl.

Typical heteroaryl(C₁₋₆)alkyl groups include furylmethyl, furylethyl, thienylmethyl, thienylethyl, oxazolylmethyl, oxazolylethyl, thiazolylmethyl, thiazolylethyl, imidazolylmethyl, imidazolylethyl, oxadiazolylmethyl, oxadiazolylethyl, thiadiazolylmethyl, thiadiazolylethyl, triazolylmethyl, triazolylethyl, tetrazolylmethyl, tetrazolylethyl, pyridinylmethyl, pyridinylethyl, pyrimidinylmethyl, pyrazinylmethyl, quinolinylmethyl and isoquinolinylmethyl.

Where a compound or group is described as “optionally substituted” one or more substituents may be present. Optional substituents are not particularly limited and may, for instance, be selected from C₁₋₆ alkyl, C₂₋₆ alkenyl, C₃₋₇ cycloalkyl, C₃₋₇ heterocycloalkyl, aryl, aryl(C₁₋₆)alkyl, heteroaryl, heteroaryl(C₁₋₆)alkyl, C₁₋₆ alkoxy, aryloxy, aryl(C₁₋₆)alkoxy, heteroaryloxy, heteroaryl(C₁₋₆)alkoxy, amino, nitro, halo, hydroxy, carboxy, formyl, cyano and trihalomethyl groups. Furthermore, optional substituents may be attached to the compounds or groups which they substitute in a variety of ways, either directly or through a connecting group of which the following are examples: amine, amide, ester, ether, thioether, sulphonamide, sulphamide, sulphoxide, urea, thiourea and urethane. As appropriate an optional substituent may itself be substituted by another substituent, the latter being connected directly to the former or through a connecting group such as those exemplified above.

Where the compounds according to the invention have at least one asymmetric centre, they may accordingly exist as enantiomers. Where the compounds according to the invention possess two or more asymmetric centres, they may additionally exist as diastereoisomers. It is to be understood that all such isomers and mixtures thereof in any proportion are encompassed within the scope of the present invention.

Where the moiety Z in the compounds of formula (I) above represents optionally substituted C₂₋₆ alkynyl, this is suitably an optionally substituted ethynyl group. A typical substituent on the C₂₋₆ alkynyl group is tri(C₁₋₆)alkylsilyl, especially trimethylsilyl. In this context, a typical value for the moiety Z is trimethylsilylethynyl.

Where Z represents an optionally substituted aryl or heteroaryl moiety, it may suitably be selected from phenyl, thienyl, oxazolyl, thiazolyl, furyl, isoquinolinyl, indolyl, isoxazolyl, pyrazolopyrimidinyl and pyrazinyl, any of which groups may be optionally substituted. Particular values of Z include phenyl, thienyl, thiazolyl and furyl, any of which groups may be optionally substituted. These groups may be joined to the 5-position of the pyridinone nucleus at any available position of the aryl or heteroaryl ring. However, connection at certain positions may be preferred and this is considered in some more detail below.

Preferred optional substituents on the aryl or heteroaryl group Z may be selected from a wide variety of groups. For instance, they may be simple, relatively small groups such as halogen (especially fluorine, chlorine and bromine), hydroxy, —NO₂, —NH₂, formyl, C₂₋₆ alkylcarbonyl, —CO₂H, C₂₋₆ alkoxycarbonyl, C₁₋₆ alkyl (especially methyl), C₂₋₆ alkenyl, C₂₋₆ alkynyl, —CN, C₁₋₆ alkoxy (especially methoxy), C₁₋₆ alkylthio (especially methylthio), C₁₋₆ alkylsulfinyl (especially methylsulfinyl) or C₁₋₆ alkylsulfonyl (especially methylsulfonyl). As appropriate any of these substituents may be substituted by one or more of the others. However, in general at least one substituent is a group of formula (II):

—X—R² (II)

where R² is a generally hydrophobic moiety containing one or more, but generally at least 3, preferably 4 to 20, particularly 4 to 14, carbon atoms. Preferably, R² includes one or more of the following groups, any of which may, optionally, be substituted: aryl, aryl(C₁₋₆)alkyl, C₃₋₇ cycloalkyl, C₁₋₆ alkyl (especially branched C₁₋₆ alkyl), heteroaryl, heteroaryl(C₁₋₆)alkyl, C₃₋₇ heterocycloalkyl and C₂₋₆ alkenyl. The group X is preferably selected from —NH—SO₂—, —NH—SO₂—NH—, —CH₂—SO₂—, —SO₂—NH—, —NH—CO—NH—, —NH—CS—NH—, —NH—CO—O—, —NH—CO—, —CO—NH—, —NH—CO—NH—SO₂—, —NH—CO—NH—CO—, —O—, —S—, —SO—, —SO₂—, —NH—, —CH₂—, —CH₂O— and —CH₂S—.

The hydrogen atom of any NH group may, optionally, be replaced by a C₁₋₆ alkyl group.

Particular values of R¹ include hydrogen, methyl, ethyl, morpholinylethyl, dimethylaminoethyl, acetoxymethyl, pivaloyloxymethyl and 1-(cyclohexyloxycarbonyloxy)ethyl.

Specific values of R¹ include hydrogen, methyl and ethyl.

In one embodiment, R¹ represents hydrogen.

One illustrative sub-class of compounds in accordance with the invention is represented by formula (III) below:

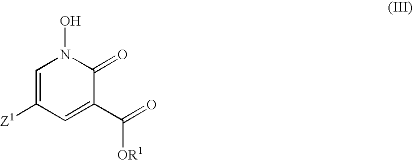

wherein

Z¹ represents optionally substituted aryl; and

R¹ is as defined above.

For instance, examples of compounds within this class are those of formula (IV):

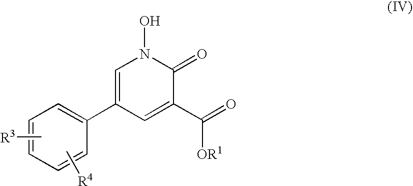

wherein

R¹ is as defined above; and

each of R³ and R⁴ may independently be selected from H or a substituent group.

Preferably, one of R³ and R⁴ is hydrogen, while the other is a substituent. Where a substituent is present it may be at any of the 2-, 3- or 4-positions—i.e. ortho, meta or para to the pyrimidinone nucleus. However, where a single substituent is present, substitution at the ortho or meta positions is preferred.

The substituents R³ and R⁴ may be selected from a wide variety of groups. For instance, they may be simple, relatively small groups such as halogen (especially fluorine, chlorine and bromine), hydroxy, —NO₂, —NH₂, formyl, C₂₋₆ alkylcarbonyl, —CO₂H, C₂₋₆ alkoxycarbonyl, C₁₋₆ alkyl (especially methyl), C₂₋₆ alkenyl, C₂₋₆ alkynyl, —CN, C₁₋₆ alkoxy (especially methoxy), C₁₋₆ alkylthio (especially methylthio), C₁₋₆ alkylsulfinyl (especially methylsulfinyl) or C₁₋₆ alkylsulfonyl (especially methylsulfonyl). As appropriate any of these substituents may be substituted by one or more of the others.

Although some such compounds are of high activity, it is generally preferable that substituent R³ and/or R⁴ include a relatively hydrophobic group R² which is bonded to the phenyl group through a linkage X. In this case the substituents R³ and/or R⁴ may be represented by the formula (II):

—X—R² (II)

where R² and X are as defined above.

For instance, examples of preferred classes of compound are those in which a single ortho or meta substituent is present, and that substituent is selected from the following formulae (V), (VI), (VII), (VIII) and (IX):

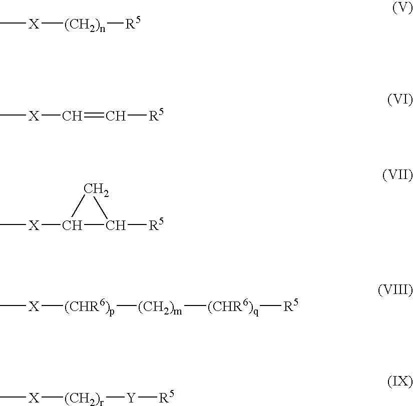

wherein

n is zero or an integer from 1 to 6, and preferably is from zero to 3, especially 0 or 1;

m is zero or an integer from 1 to 6, but preferably is 0 or 1;

each of p and q is independently 0 or 1, but preferably they are not simultaneously 1;

r is an integer from 1 to 6, preferably 1;

R⁶ is an optionally substituted aryl, heteroaryl, C₃₋₇ cycloalkyl, C₃₋₇ heterocycloalkyl or branched C₁₋₆ alkyl group;

each R⁶ is independently a C₁₋₆ alkyl group (especially methyl), a C₃₋₇ cycloalkyl group, an optionally substituted aryl group (especially phenyl), hydroxy or hydroxy(C₁₋₆)alkyl (especially hydroxymethyl), any of which may be optionally etherified, or —NH₂, optionally protonated, alkylated or derivatised as a urethane group; and

Y is selected from —O—, —S— and —NH—.

In each of the formulae (V) to (IX) the linkage X may be any of the X groups specified above.

Among the groups X, the sulfonamide (—NH—SO₂—), urea (—NH—CO—NH—), urethane (—NH—CO—O—) and amide (—NH—CO—) groups are favoured. A particular value of X is —NH—CO—NH—SO₂—.

In a specific embodiment, X represents —NH—CO—NH— or —NH—CO—.

The group R⁵ is preferably an aryl or heteroaryl group, of which optionally substituted phenyl, naphthyl, thienyl, benzothienyl, pyridyl, quinolyl and thiazolyl are particularly preferred examples. Each of these may, optionally, be substituted by another optionally substituted aryl or heteroaryl group of the same or different type.

Typical compounds of formula (IV) are specifically exemplified herein as Examples 1 to 5. All those compounds have IC₅₀ values no greater than 100 μM when measured in the assay described below.

Another illustrative sub-class of compounds in accordance with the invention is represented by formula (X) below:

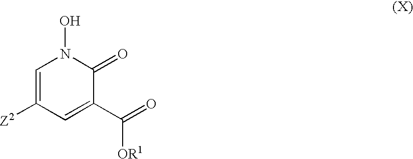

wherein

Z² represents optionally substituted heteroaryl; and

R¹ is as defined above.

Particular values of Z² include thienyl, thiazolyl and furyl, especially thienyl, any of which groups may be optionally substituted.

Preferred compounds in this sub-class are those in which the heteroaryl group Z² is unsubstituted, or carries a single substituent R⁷, as defined infra.

A favoured subset of the compounds of formula (X) is represented by formula (XI) below:

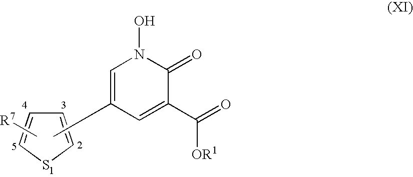

wherein

R¹ is as defined above; and

R⁷ is as defined infra.

The pyridinone nucleus and the R⁷ substituent may be at any position on the thiophene ring. However, it is preferred that when the pyridinone is at position 2 on the thiophene ring, then substituent R⁷ is at the 3-position, substitution at the 4 or 5-positions being less preferred. When the pyridinone group is at the 3-position of the thiophene ring, then R⁷ is preferably at the 2- or 4-position of the thiophene ring, more preferably at the 4-position. In summary, favoured compounds in accordance with the present invention are represented by formula (XII) and (XIII) below:

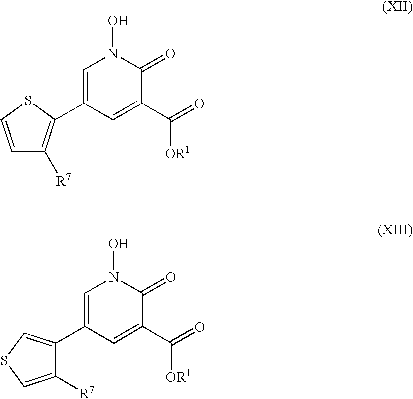

wherein

R¹ is as defined above; and

R⁷ is as defined infra.

Substituent R⁷ may be selected from a wide variety of groups. For instance, like substituents R³ and R⁴ discussed above it may be a simple, relatively small group such as halogen (especially fluorine, chlorine and bromine), hydroxy, —NO₂, —NH₂, formyl, C₂₋₆ alkylcarbonyl, —CO₂H, C₂₋₆ alkoxycarbonyl, C₁₋₆ alkyl (especially methyl), C₁₋₆ alkenyl, C₂₋₆ alkynyl, —CN, C₁₋₆ alkoxy (especially methoxy), C₁₋₆ alkylthio (especially methylthio), C₁₋₆ alkylsulfinyl (especially methylsulfinyl) or C₁₋₆ alkylsulfonyl (especially methylsulfonyl). As appropriate any of these substituents may be substituted by one or more of the others.

More preferably, however, R⁷ includes a relatively hydrophobic group which is bonded to the thienyl group through a linkage X. In this case, the group R⁷ may be represented by the formula (II):

—X—R² (II)

where X and R² are as defined above.

Preferred X groups are amide, sulphonamide, urea and urethane linkages. A particularly preferred X group is —NH—CO—NH—SO₂—. Preferred R² groups are those shown in formulae (V) to (IX) already discussed above, and which include a group R⁵. Advantageously, R² is naphthyl.

Preferred R⁵ groups are aromatic groups, especially phenyl, naphthyl, thienyl, pyridyl, benzothienyl, indolyl, benzimidazolyl and oxazolyl groups. When R⁵ comprises fused aromatic rings, the connection to the remainder of the R² group may be through any ring.

Preferred optional substituents on R⁵, especially in the case where R⁵ is an aryl group, include halogen (e.g. fluorine, chlorine and/or bromine), nitro (—NO₂), C₁₋₆ alkyl (especially methyl), C₁₋₆ alkoxy (especially methoxy), trifluoromethyl and aryl (especially phenyl).

Suitably, n is zero.

Suitably, R⁵ is naphthyl.

In another aspect, the invention provides the use of a compound of formula (I) as defined above, or a tautomer thereof, or a pharmaceutically acceptable salt thereof, for the manufacture of a medicament for treatment or prevention of infection by hepatitis C virus in a human or animal.

A further aspect of the invention provides a pharmaceutical composition comprising a compound of formula (I) as defined above, or a tautomer thereof, or a pharmaceutically acceptable salt thereof, in association with a pharmaceutically acceptable carrier. The composition may be in any suitable form, depending on the intended method of administration. It may for example be in the form of a tablet, capsule or liquid for oral administration, or of a solution or suspension for administration parenterally.

The pharmaceutical compositions optionally also include one or more other agents for the treatment of viral infections such as an antiviral agent, or an immunomodulatory agent such as α-, β- or γ-interferon.

In a further aspect, the invention provides a method of inhibiting hepatitis C virus polymerase and/or of treating or preventing an illness due to hepatitis C virus, the method involving administering to a human or animal (preferably mammalian) subject suffering from the condition a therapeutically or prophylactically effective amount of the pharmaceutical composition described above or of a compound of formula (I) as defined above, or a tautomer thereof, or a pharmaceutically acceptable salt thereof. “Effective amount” means an amount sufficient to cause a benefit to the subject or at least to cause a change in the subject's condition.

The dosage rate at which the compound is administered will depend on a variety of factors including the activity of the specific compound employed, the metabolic stability and length of action of that compound, the age of the patient, body weight, general health, sex, diet, mode and time of administration, rate of excretion, drug combination, the severity of the particular condition and the host undergoing therapy. Suitable dosage levels may be of the order of 0.02 to 5 or 10 g per day, with oral dosages two to five times higher. For instance, administration of from 10 to 50 mg of the compound per kg of body weight from one to three times per day may be in order. Appropriate values are selectable by routine testing. The compound may be administered alone or in combination with other treatments, either simultaneously or sequentially. For instance, it may be administered in combination with effective amounts of antiviral agents, immunomodulators, anti-infectives or vaccines known to those of ordinary skill in the art. It may be administered by any suitable route, including orally, intravenously, cutaneously and subcutaneously. It may be administered directly to a suitable site or in a manner in which it targets a particular site, such as a certain type of cell. Suitable targeting methods are already known.

An additional aspect of the invention provides a method of preparation of a pharmaceutical composition, involving admixing at least one compound of formula (I) as defined above, or a tautomer thereof, or a pharmaceutically acceptable salt thereof, with one or more pharmaceutically acceptable adjuvants, diluents or carriers and/or with one or more other therapeutically or prophylactically active agents.

The compounds according to the present invention may be prepared by a process which comprises reacting a compound of formula (XIV) with a compound of formula (XV):

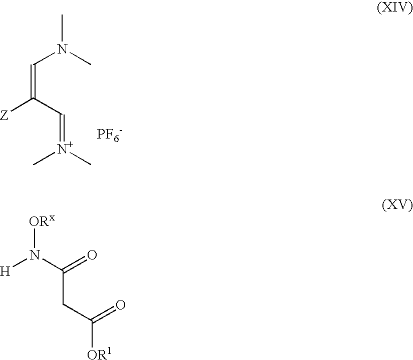

wherein Z and R¹ are as defined above, and R^{x} represents a hydroxy-protecting group; followed by removal of the hydroxy-protecting group R^{x}.

The reaction between compounds (XIV) and (XV) is conveniently accomplished at an elevated temperature in the presence of a base such as potassium tert-butoxide, typically in a solvent such as tetrahydrofuran.

Typical values for the hydroxy-protecting group R^{x} include tert-butyl and benzyl, in which case the hydroxy-protecting group R^{x} can be removed by treatment with a strong acid, e.g. hydrochloric acid, or by catalytic hydrogenation.

The intermediates of formula (XIV) above may be prepared from the corresponding compound of formula Z-CH₂—CO₂H by treatment with phosphorus oxychloride and N,N-dimethylformamide at an elevated temperature (e.g. 70° C.); followed by treatment with hexafluorophosphoric acid in the presence of a base such as sodium hydroxide.

The intermediates of formula (XV) above may be prepared by reacting a compound of formula H₂N—OR^{x} with a compound of formula (XVI):

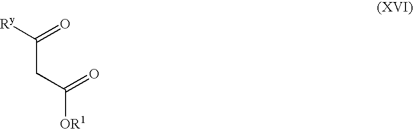

wherein R¹ and R^{x} are as defined above, and R^{y} represents hydroxy or a halogen atom, e.g. chloro.

In another procedure, the compounds according to the present invention may be prepared by a process which comprises oxidizing a compound of formula (XVII):

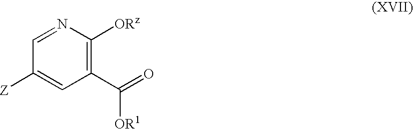

wherein Z and R¹ are as defined above, and R^{z} represents C₁₋₆ alkyl, e.g. methyl; followed by cleavage of the R^{z} moiety.

The oxidation of compound (XVII) is conveniently accomplished by treatment with a peracid, e.g. trifluoroperacetic acid.

Cleavage of the R^{z} moiety may conveniently be effected by treatment with a strong acid, e.g. hydrochloric acid.

The intermediates of formula (XVII) above may be prepared by reacting a compound of formula (XVIII) with a compound of formula (XIX):

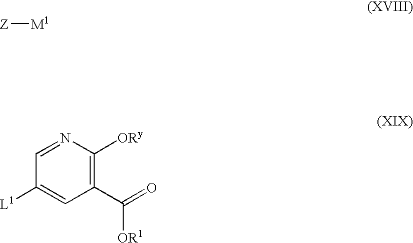

wherein Z, R¹ and R^{y} are as defined above, L¹ represents a suitable leaving group, and M¹ represents a boronic acid moiety —B(OH)₂, or a cyclic ester thereof formed with an organic diol, e.g. pinacol, 1,3-propanediol or neopentyl glycol; in the presence of a transition metal catalyst.

The leaving group L¹ is typically a halogen atom, e.g. bromo.

The transition metal catalyst of use in the reaction between compounds (XVII) and (XIX) is suitably tetrakis(triphenylphosphine)-palladium(0). The reaction is conveniently carried out at an elevated temperature in a solvent such as toluene, tetrahydrofuran, 1,4-dioxane or N,N-dimethylformamide, typically in the presence of potassium phosphate, sodium carbonate, cesium carbonate or copper(I) iodide.

Where they are not commercially available, the starting materials of formula (XVI), (XVIII) and (XIX) may be prepared by methods analogous to those described in the accompanying Examples, or by standard methods well known from the art.

It will be understood that any compound of formula (I) initially obtained from any of the above processes may, where appropriate, subsequently be elaborated into a further compound of formula (I) by techniques known from the art. For example, a compound of formula (I) wherein the moiety Z is substituted by a simple, relatively small group as specified supra may be converted into the corresponding compound wherein Z is substituted by a group of formula (II) as defined above by means of procedures analogous to those described in many of the accompanying Examples. By way of specific example, a compound of formula (I) wherein Z is substituted by nitro may be converted into the corresponding compound wherein Z is substituted by amino by means of catalytic hydrogenation. A compound of formula (I) wherein R¹ represents hydrogen may be converted into the corresponding compound wherein R¹ is other than hydrogen by means of conventional esterification procedures, e.g. by treatment with the appropriate alcohol of formula R¹—OH in the presence of a mineral acid such as hydrochloric acid. A compound of formula (I) wherein R¹ is other than hydrogen may be converted into the corresponding compound wherein R¹ is hydrogen by means of standard saponification techniques, e.g. by treatment with an alkaline reagent such as sodium hydroxide or lithium hydroxide.

Where a mixture of products is obtained from any of the processes described above for the preparation of compounds according to the invention, the desired product can be separated therefrom at an appropriate stage by conventional methods such as preparative HPLC; or column chromatography utilising, for example, silica and/or alumina in conjunction with an appropriate solvent system.

Where the above-described processes for the preparation of the compounds according to the invention give rise to mixtures of stereoisomers, these isomers may be separated by conventional techniques such as preparative chromatography. The novel compounds may be prepared in racemic form, or individual enantiomers may be prepared either by enantiospecific synthesis or by resolution. The novel compounds may, for example, be resolved into their component enantiomers by standard techniques such as preparative HPLC, or the formation of diastereomeric pairs by salt formation with an optically active acid, such as (−)-di-p-toluoyl-d-tartaric acid and/or (+)-di-p-toluoyl-l-tartaric acid, followed by fractional crystallization and regeneration of the free base. The novel compounds may also be resolved by formation of diastereomeric esters or amides, followed by chromatographic separation and removal of the chiral auxiliary.

During any of the above synthetic sequences it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 3rd edition, 1999. The protecting groups may be removed at a convenient subsequent stage using methods known from the art.

The following Examples illustrate the preparation of compounds according to the invention.

The compounds in accordance with this invention are potent inhibitors of HCV polymerase. The IC₅₀ values in μM of these compounds can be measured in the following way.

### Test for Inhibition of Hepatitis C Virus RdRp

WO 96/37619 describes the production of recombinant HCV RdRp from insect cells infected with recombinant baculovirus encoding the enzyme. The purified enzyme was shown to possess in vitro RNA polymerase activity using RNA as template. The reference describes a polymerisation assay using poly(A) as a template and oligo(U) as a primer. Incorporation of tritiated UTP is quantified by measuring acid-insoluble radioactivity. The present inventors have employed this assay to, screen the compounds of the accompanying Examples as inhibitors of HCV RdRp.

Incorporation of radioactive UMP was measured as follows. The standard reaction (100 μl) was carried out in a buffer containing 20 mM tris/HCl pH 7.5, 5 mM MgCl₂, 1 mM DTT, 50 mM NaCl, 1 mM EDTA, 2OU Rnasin (Promega), 0.05% Triton X-100, 1 μCi [³H]-UTP (40 Ci/mmol, NEN), 10 μM UTP and 10 μg/ml poly(A). Oligo(U)₁₂ (1 μg/ml, Genset) was added as a primer. The final NSSB enzyme concentration was 20 nM. After 1 h incubation at 22° C. the reaction was stopped by adding 100 μl of 20% TCA and applying samples to DE81 filters. The filters were washed thoroughly with 5% TCA containing 1M Na₂HPO₄/NaH₂PO₄, pH 7.0, rinsed with water and then ethanol, air dried, and the filter-bound radioactivity was measured in the scintillation counter. By carrying out the reaction in the presence of various concentrations of each test compound it was possible to determine IC₅₀ values for each compound utilizing the formula:

% residual activity=100/(1+[I]/IC₅₀)^{s}

where [I] is the inhibitor concentration and “s” is the slope of the inhibition curve.

The compounds of the accompanying Examples were tested in the above assay, and all were found to possess an IC₅₀ value of 100 μM or less.

### EXAMPLE 1

### 1-Hydroxy-2-oxo-5-phenyl-1,2-dihydropyridine-3-carboxylic acid

### a) 5-Bromo-2-hydroxynicotinic acid

This compound was prepared according to the procedure of Y. S. Lo (Synthetic Communications, 1989, 553). Bromine (0.77 eq) was added dropwise at 0° C. to a stirred solution of 50% NaOH (2.4 eq) in water (1 M solution). After 5 min, 50% NaOH (3 eq) followed by solid 2-hydroxynicotinic acid (1 eq) was added to the mixture and the resulting solution was stirred at 50° C. After 20 h, a solution prepared by adding bromine (0.38 eq) to 50% NaOH (1.2 eq) in water (1 M solution) was added to the reaction mixture and stirring continued for another 24 h at 50° C. After that time the reaction mixture was cooled to 0° C. and acidified to pH 2 with concentrated hydrochloric acid to allow the formation of a solid which was isolated by filtration, washed with warm water/isopropanol (3:1), then with diethyl ether and dried to afford 5-bromo-2-hydroxynicotinic acid (87%) as an off-white solid. δ_{H} (400 MHz; DMSO) 8.25 (1H, d, J 2.7), 8.33 (1H, d, J 2.7), 13.84 (2H, bs); δ_{C} (400 MHz; DMSO) 99.45, 117.97, 142.01, 147.42, 163.22, 163.88; m/z (ES⁻) 218-216 (M−H)⁻.

### b) Methyl 5-bromo-2-methoxynicotinate

A solution of 5-bromo-2-hydroxynicotinic acid (1 eq) and N,N-dimethylformamide (1 eq) in thionyl chloride (0.88 M solution) was refluxed for 2 h. Thionyl chloride was evaporated and the residue suspended in anhydrous dichloromethane (0.7 M) and anhydrous methyl alcohol (35 eq) was added dropwise. The resulting mixture was refluxed for 1 h, then evaporated in vacuo to obtain an oily residue which was dissolved in dry methanol and added to a stirred solution of sodium methoxide (1.3 eq) in the same solvent (1 M solution). The reaction mixture was stirred for 3 h at room temperature then neutralized by addition of a few drops of acetic acid and extracted into ethyl acetate. The organic layer was washed with a saturated solution of aqueous sodium hydrogencarbonate, brine, dried over sodium sulfate and evaporated in vacuo. The residue was crystallized from hot diethyl ether to afford methyl 5-bromo-2-methoxynicotinate (44%) as beige, shiny crystals. δ_{H} (400 MHz; CDCl₃) 3.89 (3H, s), 4.01 (3H, s), 8.23 (1H, d, J 2.5), 8.33 (1H, d, J 2.5); m/z (ES⁺) 247 (M⁺+H).

### c) Methyl 2-methoxy-5-phenylnicotinate

Methyl 5-bromo-2-methoxynicotinate (1 eq), phenylboronic acid (1.5 eq), K₃PO₄.H₂O (2 eq) and tetrakis(triphenylphosphine)palladium (0.05 eq) in toluene (0.17 M solution) were placed in a Schlenk tube, purged with 2 vacuum/argon cycles and heated at reflux overnight. The cooled reaction mixture was diluted with ethyl acetate, washed with water (2×) and brine, then dried over sodium sulfate and evaporated in vacuo. The crude residue was purified by flash chromatography (SiO₂, petroleum ether/ethyl acetate 8:1) to afford methyl 2-methoxy-5-phenylnicotinate (93%) as a yellowish oil. δ_{H} (400 MHz; CDCl₃) 3.93 (3H, s), 4.10 (3H, s), 7.38 (1H, t, J 7.2), 7.46 (2H, t, J 7.2), 7.55 (2H, d, J 7.5), 8.38 (1H, d, J 1.9), 7.54 (1H, t, J 1.9); m/z (ES⁺) 244 (M⁺+H).

### d) Methyl 2-methoxy-5-phenylnicotinate 1-oxide

A solution of methyl 2-methoxy-5-phenylnicotinate (1 eq) in dichloromethane (0.3 M solution) was added at room temperature to a preformed solution of trifluoroperacetic acid (5 eq) and urea in the same solvent. The peracid was prepared by adding an equimolar amount of trifluoroacetic anhydride to a suspension of urea/H₂O₂ complex in dichloromethane at 0° C. and stirring the resulting suspension for 10 min at room temperature. After being stirred for 2 h at room temperature, the reaction mixture was treated again with trifluoroperacetic acid (5 eq), and after another hour it was diluted with chloroform, thoroughly washed with saturated sodium thiosulfate, dried over sodium sulfate and evaporated. The crude residue was purified by medium-pressure RP column (Lobar-C18-Merck, water/acetonitrile 1:1) affording methyl 2-methoxy-5-phenylnicotinate 1-oxide (17%) as a yellow powder. δ_{H} (400 MHz; DMSO) 3.97 (3H, s), 4.30 (3H, s), 7.36-7.60 (5H, m), 7.95 (1H, d, J 2.5), 8.62 (1H, d, J 2.5); m/z (ES⁺) 260 (M⁺+H).

### e) 1-Hydroxy-2-oxo-5-phenyl-1,2-dihydropyridine-3-carboxylic acid

The foregoing compound (1 eq) was refluxed overnight in hydrochloric acid (6 N, 0.03 M solution). The reaction mixture was allowed to cool to room temperature, diluted with water/acetonitrile (1:1) and purified by RP-HPLC on a Prep NOVAPAK (Waters) C18 Cartridge Column (7 micron, 25×100 mm; Flow: 10 ml/min; Gradient: A: H₂O+0.05% TFA; B: MeCN+0.05% TFA; 70% A isocratic for 2 min then linear to 30% A in 5 min). The title compound was obtained after lyophilization. δ_{H} (400 MHz; DMSO) 7.37 (1H, t, J 7.3), 7.46 (2H, t, J 7.3), 7.67 (2H, d, J 7.6), 8.49 (1H, d, J 2.6), 8.83 (1H, d, J 2.6), 13.00 (1H, bs), 14.15 (1H, bs); δ_{C} (400 MHz; DMSO-d₆) 116.93, 119.50, 125.98, 127.84, 129.03, 133.91, 138.64, 140.01, 158.93, 164.36; m/z (ES⁻) 230 (M−H).

### EXAMPLE 2

### 1-Hydroxy-5-{3-[({[1-(1-naphthyl)ethyl]amino}carbonyl)amino]phenyl}-2-oxo-1,2-dihydropyridine-3-carboxylic acid

### a) N-[(2Z)-3-(Dimethylamino)-2-(3-nitrophenyl)prop-2-enylidene]-N-methylmethanaminium hexafluorophosphate

Two modified literature procedures were used. According to a procedure by Coppola et al. (J. Heterocyclic Chem., 1974, 11, 51) and a procedure by I. W. Davies et al. (J. Org. Chem., 2000, 65, 4571), anhydrous DMF (3.7 eq) was added dropwise to neat phosphorus oxychloride (3 eq) with intermittent cooling in order to maintain the internal temperature below 30° C. The resulting mixture was stirred for 5 minutes at room temperature, then a solution of 3-nitrophenylacetic acid (1 eq) in dry DMF (2 M solution) was added dropwise over 5 min. The yellow-orange reaction mixture was stirred at 70° C. for 2 h. The cooled reaction mixture was transferred into a dropping funnel and added, concomitantly with an aqueous solution of NaOH (5 N, 47.5 eq), to a stirred solution of commercial hexafluorophosphoric acid (60% wt; 18 eq) and NaOH (5 N, 25 eq) in water (0.1 M solution), at 0° C. over 40 min. A precipitate formed, which was aged for one hour, filtered, washed with water and finally dried in vacuo over phosphorus pentoxide to afford the title compound as a light yellow solid (63%). δ_{H} (400 MHz; DMSO) 2.44 (6H, s), 3.26 (6H, s), 7.72 (1H, t, J 8.0), 7.77-7.79 (3H, m), 8.16 (1H, s), 8.28 (1H, d, J 8.0); δ_{C} (400 MHz; DMSO) 48.61, 102.37, 123.42, 126.16, 129.57, 134.49, 138.32, 147.29, 162.85; m/z (ES⁺) 248 (M⁺+H).

### b) Methyl 3-(tert-butoxyamino)-3-oxopropanoate

To a suspension of O-tert-butylhydroxylamine hydrochloride (1.1 eq) and DIPEA (2.2 eq) in anhydrous THF (0.7 M solution), cooled to 0° C., was added a solution of methyl 3-chloro-3-oxopropionate (1 eq) in the same solvent (3 M solution). The resulting suspension was stirred at room temperature for 24 h. The solid was filtered off and the remaining solution was diluted with ethyl acetate and washed with hydrochloric acid (1 N). The aqueous layer was extracted again with ethyl acetate (2×) and with chloroform (2×). The combined organic layers were washed with brine and dried over sodium sulfate. Evaporation afforded the title compound as a colorless oil, which solidified upon standing. δ_{H} (400 MHz; DMSO) 1.15 (9H, s), 3.16 (2H, s), 3.62 (3H, s), 10.52 (1H, s).

### c) Methyl 1-tert-butoxy-5-(3-nitrophenyl)-2-oxo-1,2-dihydropyridine-3-carboxylate

A solution of methyl 3-(tert-butoxyamino)-3-oxopropanoate (1 eq) in anhydrous THF (0.2 M) was treated with solid potassium tert-butoxide (1.1 eq) at 0° C. The resulting solution was stirred for 10 min at 0° C., then for 1 h at room temperature and treated with the title compound from step b (1.3 eq), which was added in one portion. The suspension thus obtained was stirred for 4 h at 45° C., then diluted with ethyl acetate and washed with hydrochloric acid (1 N, 3×) and brine. Drying over sodium sulfate and evaporation gave the crude product, which after purification by flash chromatography (silica gel, petroleum ether/ethyl acetate 1:2, containing 1% of MeOH) afforded methyl 1-tert-butoxy-5-(3-nitrophenyl)-2-oxo-1,2-dihydropyridine-3-carboxylate (67%) as a light yellow solid. δ_{H} (400 MHz; DMSO-d₆) 1.40 (9H, s), 3.82 (3H, s), 7.74 (1H, t, J 8.1), 8.11 (1H, d, J 8.1), 8.19 (1H, d, J 8.1), 8.43 (1H, d, J 2.7), 8.45 (1H, s), 8.67 (1H, d, J 2.7); δ_{C} (400 MHz; DMSO-d₆) 29.31, 54.48, 90.51, 116.01, 122.94, 124.23, 124.36, 132.76, 134.86, 138.71, 143.79, 144.76, 150.76, 158.15, 167.51; m/z (ES⁺) 347 (M⁺+H).

### d) Methyl 5-(3-aminophenyl)-1-tert-butoxy-2-oxo-1,2-dihydro-pyridine-3-carboxylate

A solution of methyl 1-tert-butoxy-5-(3-nitrophenyl)-2-oxo-1,2-dihydropyridine-3-carboxylate in methyl alcohol (0.36 M) was hydrogenated at atmospheric pressure over Lindlar's catalyst (20% w/w) for 5 h. Removal of the catalyst by filtration, followed by evaporation of the solvent in vacuo, gave methyl 5-(3-aminophenyl)-1-tert-butoxy-2-oxo-1,2-dihydropyridine-3-carboxylate (95%) as an off-white solid. δ_{H} (300 MHz; DMSO) 1.38 (9H, s), 3.80 (3H, s), 5.20 (2H, bs), 6.55 (1H, d, J 7.8), 6.71 (1H, d, J 7.8), 6.75 (1H, s), 7.09 (1H, t, J 7.8), 8.24 (1H, d, J 2.7), 8.27 (1H, d, J 2.7); m/z (ES⁺) 317 (M⁺+H).

### e) 1-Hydroxy-5-{3-[({[1-(1-naphthyl)ethyl]amino}carbonyl)amino]phenyl}-2-oxo-1,2-dihydropyridine-3-carboxylic acid

A solution of 3-[1-hydroxy-5-(methoxycarbonyl)-6-oxo-1,6-dihydropyridin-3-yl]benzenaminium trifluoroacetate (1 eq) in anhydrous pyridine (0.1 M) was treated with 1-(1-isocyanatoethyl)naphthalene (2 eq) and the resulting solution was stirred at room temperature overnight. Pyridine was evaporated in vacuo and the residue re-dissolved in THF (0.1 M), treated with aqueous potassium hydroxide (1 N, 3 eq) and heated at 45° C. for 2 h. The reaction mixture was cooled in an ice-bath and acidified to pH=1 with hydrochloric acid (1 N). The resulting mixture was diluted with water/acetonitrile (1/1) and purified by RP-HPLC using a Prep NOVAPAK (Waters) C18 Cartridge Column (7 micron, 25×100 mm; Flow: 10 ml/min; Gradient: A: H₂O+0.05% TFA; B: MeCN+0.05% TFA; 60% A isocratic for 2 min then linear to 30% A in 8 min). After lyophilization the title compound (55%) was obtained as a colorless powder. δ_{H} (300 MHz; DMSO) 1.56 (3H, d, J 6.6), 5.67 (1H, m), 6.87 (1H, d, J 7.8), 7.20-7.24 (1H, m), 7.31-7.33 (2H, m), 7.50-7.62 (4H, m), 7.74 (1H, s), 7.85 (1H, d, J 7.8), 7.97 (1H, d, J 7.8), 8.19 (1H, d, J 8.4), 8.43 (1H, d, J 2.7), 8.55 (1H, s), 8.74 (1H, d, J 2.7); δ_{C} (300 MHz; DMSO-d₆) 22.07, 44.58, 114.84, 116.86, 117.05, 118.73, 119.64, 122.03, 123.01, 125.40, 125.52, 126.13, 127.19, 128.56, 129.45, 130.18, 133.34, 134.34, 138.49, 139.81, 140.55, 141.05, 154.19, 158.93, 164.34; m/z (ES⁺) 444 (M⁺+H).

### EXAMPLE 3

### 5-(3-{[(5-Bromothien-2-yl)carbonyl]amino}phenyl)-1-hydroxy-2-oxo-1,2-dihydropyridine-3-carboxylic acid

### a) Ethyl 3-[(benzyloxy)amino]-3-oxopropanoate

A suspension of O-benzylhydroxylamine hydrochloride (1.1 eq) and triethylamine (2.2 eq) in anhydrous THF (0.7 M solution) was treated dropwise at 0° C. with a solution of ethyl 3-chloro-3-oxopropionate (1 eq) in the same solvent (3 M solution). The resulting suspension was stirred at room temperature for 24 h. The solid was filtered off and the remaining solution diluted with ethyl acetate and washed with hydrochloric acid (1 N). The aqueous layer was extracted again with ethyl acetate (2×) and with chloroform (2×). The combined organic layers were washed with brine and dried over sodium sulfate. Evaporation afforded ethyl 3-[(benzyloxy)amino]-3-oxopropanoate (50%) as a colorless oil, which solidified upon standing. δ_{H} (300 MHz; DMSO) 1.20 (3H, t, J 6.9), 3.12 (2H, s), 4.10 (2H, q, J 6.9), 4.81 (2H, s), 7.36-7.40 (5H, m).

### b) Ethyl 1-(benzyloxy)-5-(3-nitrophenyl)-2-oxo-1,2-dihydropyridine-3-carboxylate

A solution of ethyl 3-[(benzyloxy)amino]-3-oxopropanoate (1 eq) in anhydrous THF (0.2 M) was treated with solid potassium tert-butoxide (1.1 eq) at 0° C. The resulting solution was stirred for 10 min at 0° C., then for 1 h at room temperature. The title compound from Example 2, step b (1.3 eq) was added as a solid in one portion. The resulting suspension was stirred for 4 h at 45° C., then diluted with ethyl acetate and washed with hydrochloric acid (1 N, 3×) and brine. Drying over sodium sulfate and evaporation gave a crude residue, which after flash chromatography purification (silica gel, petroleum ether/ethyl acetate 1:2, containing 1% of MeOH) afforded ethyl 1-(benzyloxy)-5-(3-nitrophenyl)-2-oxo-1,2-dihydropyridine-3-carboxylate (59%) as a light yellow solid. δ_{H} (300 MHz; DMSO-d₈) 1.33 (3H, t, J 7.2), 4.31 (2H, q, J 7.2), 5.32 (2H, s), 7.45-7.47 (3H, m), 7.59-7.61 (2H, m), 7.74 (1H, t, J 7.8), 8.07 (1H, d, J 7.4), 8.20 (1H, d, J 7.4), 8.41 (1H, s), 8.43 (1H, d, J 2.9), 8.80 (1H, d, J 2.9); m/z (ES⁺) 395 (M⁺+H).

### c) 5-(3-{[(5-Bromothien-2-yl)carbonyl]amino}phenyl)-1-hydroxy-2-oxo-1,2-dihydropyridine-3-carboxylic acid

A solution of ethyl 1-(benzyloxy)-5-(3-nitrophenyl)-2-oxo-1,2-dihydropyridine-3-carboxylate (1 eq) in MeOH/THF (1:1, 0.03 M) was hydrogenated at atmospheric pressure over Lindlar's catalyst (20% w/w) for 3 h. Removal of the catalyst by filtration, followed by evaporation of the solvent in vacuo, gave crude ethyl 5-(3-aminophenyl)-1-hydroxy-2-oxo-1,2-dihydropyridine-3-carboxylate, which was dissolved in dichloromethane (0.2 M) and triethylamine (1.1 eq). The resulting mixture was added to a preformed solution of 5-bromothiophene-2-carboxylic acid (1 eq), BOP-Cl (1 eq) and triethylamine (1.1 eq) in dichloromethane (0.2 M). After stirring the reaction mixture overnight at room temperature, it was diluted with ethyl acetate, washed with hydrochloric acid (1 N) and brine, then dried over sodium sulfate and evaporated in vacuo. The crude residue was dissolved in tetrahydrofuran (0.3 M) and treated with aqueous potassium hydroxide (1 N, 2.2 eq) at 50° C. for 1 h. The cooled reaction mixture was diluted with water/acetonitrile (1:1) and purified by RP-HPLC using a Prep NOVAPAK (Waters) C18 Cartridge Column: (7 micron, 25×100 mm; Flow: 10 ml/min; Gradient: A. H₂O+0.05% TFA; B: MeCN+0.05% TFA; 60% isocratic for 2 min, linear to 50% A in 8 min, isocratic at 50% A for 2 min then linear again to 30% A in 4 min). The title compound (20%) was obtained as an off-white powder upon freeze-drying of the appropriate fractions. δ_{H} (400 MHz; DMSO) 7.39 (1H, d, J 3.8), 7.43-7.46 (2H, m), 7.77-7.80 (1H, m), 7.87 (1H, d, J 3.8), 7.95 (1H, s), 8.49 (1H, d, J 2.6), 8.82 (1H, d, J 2.6), 10.37 (1H, s), 13.05 (1H, bs), 14.20 (1H, bs); m/z (ES⁻) 433-435 (M, M-2H).

### EXAMPLE 4

### 5-[2-({[(2-Chlorobenzyl)amino]carbonyl}amino)phenyl]-1-hydroxy-2-oxo-1,2-dihydropyridine-3-carboxylic acid

### a) 2-(2-Nitrophenyl)-1,3-bis(dimethylamino)trimethinium hexafluorophosphate

Following essentially the procedure described in Example 2(a), 2-(2-nitrophenyl)-1,3-bis(dimethylamino)trimethinium hexafluorophosphate (40%) was obtained as a light yellow solid. δ_{H} (300 MHz; DMSO) 2.42 (6H, s), 3.28 (6H, s), 7.57 (1H, d, J 7.2), 7.74-7.81 (4H, m), 8.10 (1H, d, J 7.4); m/z (ES⁺) 248 (M⁺+H).

### b) Methyl 1-tert-butoxy-5-(2-nitrophenyl)-2-oxo-1,2-dihydropyridine-3-carboxylate

A solution of methyl 3-(tert-butoxyamino)-3-oxopropanoate (1 eq), prepared as described in Example 2(b), in anhydrous THF (0.2 M) was treated with solid potassium tert-butoxide (1.1 eq) at 0° C. The resulting solution was stirred for 10 min at 0° C., then for 1 h at room temperature, and finally treated with 2-(2-nitrophenyl)-1,3-bis(dimethylamino)-trimethinium hexafluorophosphate (1.3 eq) in one portion. The suspension thus obtained was stirred for 6 h at 45° C., then diluted with ethyl acetate and washed with hydrochloric acid (1 N, 3×) and with brine. Drying over sodium sulfate and evaporation gave a crude residue which after flash chromatography purification (silica gel, petroleum ether/ethyl acetate 1:2, containing 1% of MeOH) afforded methyl 1-tert-butoxy-5-(2-nitrophenyl)-2-oxo-1,2-dihydropyridine-3-carboxylate (40%) as a light yellow solid. δ_{H} (400 MHz; DMSO-d₆) 1.34 (9H, s), 3.76 (3H, s), 7.61 (1H, dd, J₁ 7.6, J₂ 1.4), 7.67 (1H, dt, J₁ 8.1, J₂ 1.2), 7.80 (1H, dt, J₁ 7.6, J₂ 1.2), 7.98 (1H, d, J 2.7), 8.09 (1H, dd, J₁ 8.1, J₂ 1.4), 8.30 (1H, d, J 2.7); m/z (ES⁺) 347 (M⁺+H).

### c) 2-[1-Hydroxy-5-(methoxycarbonyl)-6-oxo-1,6-dihydropyridin-3-yl]benzenaminium trifluoroacetate

A solution of methyl 1-tert-butoxy-5-(3-nitrophenyl)-2-oxo-1,2-dihydropyridine-3-carboxylate in methyl alcohol (0.05 M) was hydrogenated at atmospheric pressure over Lindlar's catalyst (20% w/w) for 3 h. Removal of the catalyst by filtration, followed by evaporation of the solvent in vacuo, gave the crude amine which was dissolved in TFA/water (95/5, 0.07 M) and stirred for 4 h at room temperature. Evaporation of the volatiles, co-evaporation with toluene and trituration with diethyl ether afforded 2-[1-hydroxy-5-(methoxycarbonyl)-6-oxo-1,6-dihydropyridin-3-yl]benzenaminium trifluoroacetate (62%) as an off-white solid. δ_{H} (300 MHz; DMSO) 3.86 (3H, s), 6.65-6.91 (2H, m), 7.03-7.18 (2H, m), 8.04 (1H, d, J 2.5), 8.55 (1H, d, J 2.5); m/z (ES⁺) 261 (M+H).

### d) 5-[2-({[(2-Chlorobenzyl)amino]carbonyl}amino)phenyl]-1-hydroxy-2-oxo-1,2-dihydropyridine-3-carboxylic acid

A solution of 2-[1-hydroxy-5-(methoxycarbonyl)-6-oxo-1,6-dihydropyridin-3-yl]benzenaminium trifluoroacetate (1 eq) in anhydrous pyridine (0.1 M) was treated with 1-chloro-2-(isocyanatomethyl)benzene (2 eq) and the resulting solution was stirred at room temperature overnight. Pyridine was then evaporated in vacuo and the residue re-dissolved in THF (0.1 M), treated with 1 N KOH (3 eq) and heated at 45° C. for 2 h. The cooled reaction mixture, after acidification to pH=1 with 1 N HCl, was diluted with water/acetonitrile (1/1) and purified by RP-HPLC using a Prep NOVAPAK (Waters) C18 Cartridge Column (7 micron, 25×100 mm; Flow: 10 ml/min; Gradient: A: H₂O+0.05% TFA; B: MeCN+0.05% TFA; 70% A isocratic for 2 min then linear to 40% A in 8 min). The title compound (25%) was obtained as a colorless powder after freeze-drying of the appropriate fractions. δ_{H} (400 MHz; DMSO) 4.29 (2H, d, J 5.9), 6.81 (1H, t, J 5.9), 7.12 (1H, t, J 7.6), 7.22-7.35 (5H, m), 7.41 (1H, dd, J₁ 7.6, J₂ 1.9), 7.79 (1H, d, J 7.6), 7.94 (1H, s), 8.18 (1H, d, J 2.5), 8.52 (1H, d, J 2.5), 13.02 (1H, bs), 14.25 (1H, bs); m/z (ES⁻) 412 (M−H).

### EXAMPLE 5

### 1-Hydroxy-5-(2-nitrophenyl)-2-oxo-1,2-dihydropyridine-3-carboxylic acid

A suspension of methyl 1-tert-butoxy-5-(2-nitrophenyl)-2-oxo-1,2-dihydropyridine-3-carboxylate (see Example 4) was refluxed in hydrochloric acid (6 N, 0.03 M solution) for 45 min. The reaction mixture turned first homogeneous and then a colorless solid precipitated. After being cooled to room temperature, the solid was filtered off and washed with water (5×) and diethyl ether (3×), and then dried in vacuo to afford 1-hydroxy-5-(2-nitrophenyl)-2-oxo-1,2-dihydropyridine-3-carboxylic acid (20%). δ_{H} (400 M; DMSO) 7.64 (1H, dd, J₁ 7.6, J₂ 1.5), 7.69 (1H, dt, J₁ 8.2, J₂ 1.5), 7.82 (1H, dt, J₁ 7.6, J₂ 1.2), 8.13 (1H, dd, J₁ 8.2, J₂ 1.2), 8.18 (1H, d, J 2.5), 8.69 (1H, d, J 2.5), 13.01 (1H, bs), 13.95 (1H, bs); m/z (ES⁻) 275 (M⁺−H).

1. A compound of formula (I), or a pharmaceutically acceptable salt thereof: 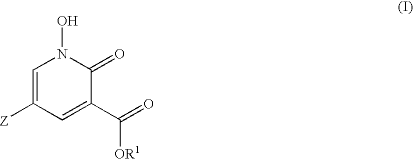
wherein
Z represents C₂₋₆ alkynyl, aryl or heteroaryl, any of which groups may be optionally substituted; and
R¹ represents hydrogen, C₁₋₆ alkyl, C₃₋₇ heterocycloalkyl(C₁₋₆)alkyl, di(C₁₋₆)alkylamino(C₁₋₆)alkyl, C₂₋₆ alkylcarbonyloxy(C₁₋₆)alkyl or C₃₋₇ cycloalkoxycarbonyloxy(C₁₋₆)alkyl.

2. A compound as claimed in claim 1 wherein Z represents optionally substituted C₂₋₆ alkynyl.

3. A compound as claimed in claim 1 wherein Z represents an optionally substituted aryl or heteroaryl moiety.

4. A compound as claimed in claim 1 wherein
R¹ is hydrogen, methyl, ethyl, morpholinylethyl, dimethylaminoethyl, acetoxymethyl, pivaloyloxymethyl or 1-(cyclohexyloxycarbonyloxy)ethyl.

5. A compound as claimed in claim 1 of formula (III): 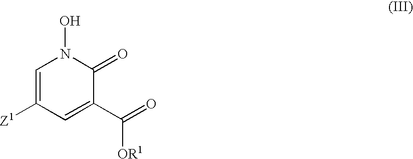
wherein
Z¹ represents optionally substituted aryl.

6. A compound according to claim 5 of formula (IV): 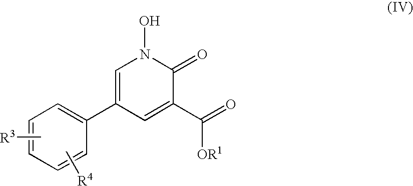
wherein
each of R³ and R⁴ is independently H or a substituent group.

7. A compound as claimed in claim 1 of formula (X): 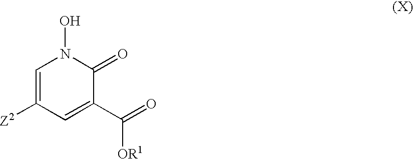
wherein
Z² represents optionally substituted heteroaryl.

8. A compound as claimed in claim 7 of formula (XI) below: 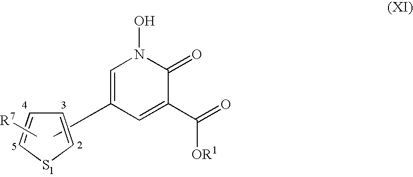
wherein
R⁷ is selected from halogen, hydroxy, —NO₂, —NH₂, formyl, C₂₋₆ alkylcarbonyl, —CO₂H, C₂₋₆ alkoxycarbonyl, C₁₋₆ alkyl, C₁₋₆ alkenyl, C₂₋₆ alkynyl, —CN, C₁₋₆ alkoxy, C₁₋₆ alkylthio, C₁₋₆ alkylsulfinyl, C₁₋₆ alkylsulfonyl or a group of the formula (II):
—X—R² (II)
where X is a linkage group and R² is a hydrophobic group.

9. A compound as claimed in claim 1, which is:
1-hydroxy-2-oxo-5-phenyl-1,2-dihydropyridine-3-carboxylic acid,
1-hydroxy-5-{3-[({[1-(1-naphthyl)ethyl]amino}carbonyl)amino]phenyl}-2-oxo-1,2-dihydropyridine-3-carboxylic acid,
5-(3-{[(5-bromothien-2-yl)carbonyl]amino}phenyl)-1-hydroxy-2-oxo-1,2-dihydropyridine-3-carboxylic acid,
5-[2-({[(2-chlorobenzyl)amino]carbonyl}amino)phenyl]-1-hydroxy-2-oxo-1,2-dihydropyridine-3-carboxylic acid,
1-hydroxy-5-(2-nitrophenyl)-2-oxo-1,2-dihydropyridine-3-carboxylic acid;
or a tautomer thereof, or a pharmaceutically acceptable salt thereof.

10-11. (canceled)

12. A pharmaceutical composition comprising a compound as claimed in claim 1, or a tautomer thereof, or a pharmaceutically acceptable salt thereof, in association with a pharmaceutically acceptable carrier.

13. The pharmaceutical composition as claimed in claim 12 which further comprises one or more other agents for the treatment of viral infections.

14. A method of inhibiting hepatitis C virus polymerase and/or of treating or preventing an illness due to hepatitis C virus, the method involving administering to a human or animal (preferably mammalian) subject suffering from the condition a therapeutically or prophylactically effective amount of a compound as claimed in claim 1, or a tautomer thereof, or a pharmaceutically acceptable salt thereof.

15. A method of preparation of a pharmaceutical composition, involving admixing at least one compound as claimed in claim 1, or a tautomer thereof, or a pharmaceutically acceptable salt thereof, with one or more pharmaceutically acceptable adjuvants, diluents or carriers.

16. A process to prepare a compound as claimed in claim 1 which comprises reacting a compound of formula (XIV) with a compound of formula (XV): 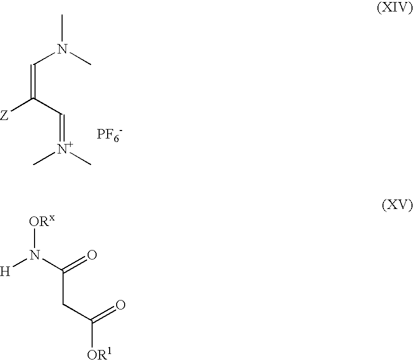
wherein R^{x} represents a hydroxy-protecting group; followed by removal of the hydroxy-protecting group R^{x}.

17. A process to prepare a compound as claimed in claim 1 which comprises oxidizing a compound of formula (XVII): 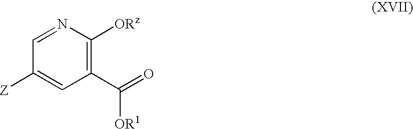
wherein R^{z} represents C₁₋₆ alkyl; followed by cleavage of the R^{z} moiety.
